(19) United States
(12) Patent Application Publication
(10) Pub. No.: US 2016/0100804 A1
(43) Pub. Date: Apr. 14, 2016

(54) METHOD FOR MEASURING A PHYSIOLOGICAL PARAMETER, SUCH AS A BIOLOGICAL RHYTHM, ON THE BASIS OF AT LEAST TWO SENSORS, AND ASSOCIATED MEASUREMENT DEVICE

(71) Applicants: CENTRE HOSPITALIER REGIONAL UNIVERSITAIRE DE LILLE, Lille (FR); SOCIETE DE RESSOURCES ET DE DEVELOPMENT POUR LES ENTREPRISES ET LES PARTICULIERS, Lille (FR)

(72) Inventors: Régis LOGIER, Marquette lez Lille (FR); Jean-Marie GROSBOIS, Lompret (FR); Alain DASSONNEVILLE, Houplines (FR); Pascal CHAUD, Santes (FR)

(21) Appl. No.: 14/766,019

(22) PCT Filed: Jan. 31, 2014
(86) PCT No.: PCT/FR2014/050173

§ 371 Date: Nov. 10, 2015

(30) Foreign Application Priority Data
Feb. 5, 2013 (FR) FR1300228

(51) Int. Cl. A61B 5/00; A61B 5/0205

(52) U.S. Cl. CPC A61B 5/7221; A61B 5/0205; A61B 5/746; A61B 5/7235

(57) ABSTRACT

A method for measuring a physiological parameter, such as a biological rhythm, on the basis of at least two sensors, and to an associated measurement device. The method includes the following steps of: measuring the physiological parameter for each sensor, allowing the generation of a series of measurements of at least two values; evaluating the level of consistency of each value from the measurement series; selecting a value from the set of values in the series as a function of the corresponding level of consistency and a so-called reference value, in order to determine a new reference value; and storing the new reference value.

The present invention relates to a method for measuring a physiological parameter, such as a biological rhythm, in addition to a device for measuring a physiological parameter for implementation of said method.

The present invention will be intended in particular to measure a physiological parameter such as the respiratory rate or heart rate of an individual in a difficult environment, for example when practising a sporting activity.

Nevertheless, although particularly intended for an application of this kind, the present invention may also be used under more conventional conditions, directly by the individual or moreover by medical staff.

Devices are known that allow measurement of heart rate or moreover respiratory rate under sporting conditions. Devices directly incorporated in the sporting equipment may be involved, such as for example in some indoor bicycles. Independent devices may also be involved, such as abdominal belts comprising a pulse sensor generally accompanied by a watch for constant display of heart rate, with these belts being conventionally used by joggers and hikers.

The existing devices generally comprise a single sensor per type of rate to be measured. Now, recording measurements by a single sensor is very difficult when the device is subject to accelerations, vibrations or furthermore impacts due to the person's movements.

In practice, it frequently occurs that when disturbance of the sensor is excessive, the measurement device in this case no longer displays any measurement until the sensor has stabilised or else it displays the last value recorded or finally it may display completely incorrect values.

As a remedy, devices comprising several sensors have been proposed in order to measure a heart rate. These devices are more reliable in that there is less probability that all the sensors will fail to perform a measurement. Nevertheless, the device does not make it possible to distinguish effectively between the consistent measurements recorded by the sensors and the incorrect measurements, such that the measurement of heartbeat or heart rate is uncertain or at the least unreliable.

It is a fact that in some cases, practice of sport must be particularly closely monitored and requires precise measurements. This is the case for example when individuals suffering from heart disorders are involved or furthermore individuals in rehabilitation performing stress resistance exercises. This is also the case when the individual is a high-level athlete whose biological parameters need to be precisely analysed and monitored.

The present invention represents an advancement in devices comprising several sensors for measuring of a physiological parameter such as in particular a biological rhythm.

The aim of the present invention is to offer a method for measuring a physiological parameter on the basis of at least two sensors, wherein the physiological parameter can be determined reliably and continuously even in a difficult data acquisition context.

Another aim of the present invention is to offer a measuring method wherein the data measured are assessed as a function of their consistency.

Another aim of the present invention is to offer a measuring method suitable for measuring heart rate and respiratory rate.

Another aim of the present invention is to offer a measurement device for implementation of the method allowing measurement of heart rate and respiratory rate.

Another aim of the present invention is to offer a measurement device incorporated in a helmet of the bicycle helmet type.

To this end, the method for measuring a physiological parameter on the basis of at least two sensors comprises, according to the invention, the following steps:
a step of measuring the physiological parameter for each sensor, allowing the generation of a series of measurements of at least two values,
a step of evaluating the level of consistency of each value of the measurement series,
a step of selecting a value from all the values of the series as a function of their respective level of consistency on the one hand and a so-called reference value on the other hand in order to determine a new reference value,
a step of storing the new reference value.

The invention also aims to protect a device for measuring a physiological parameter such as a biological rhythm, wherein said device comprises at least two sensors for measuring a physiological parameter, means of filtering the signals issued by the sensors, means of processing the measured data, means of storing the data and means of displaying the selected value.

The present invention will be better understood on reading below a detailed example of embodiment, provided by way of a non-restrictive example and with reference to the appended figures, among which:

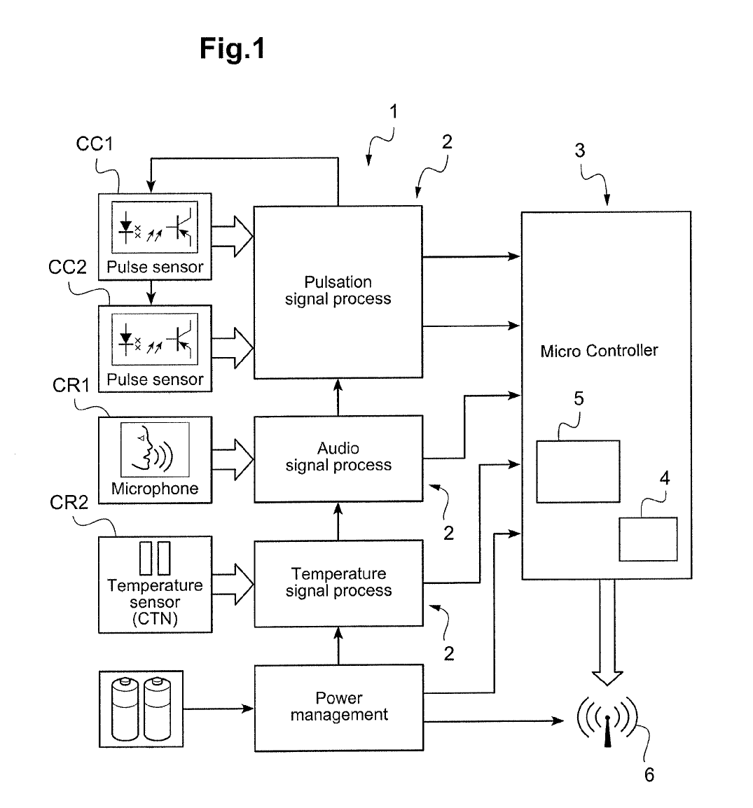

FIG. 1 represents an example of embodiment, in diagrammatic form, of the measurement device in accordance with the present invention,

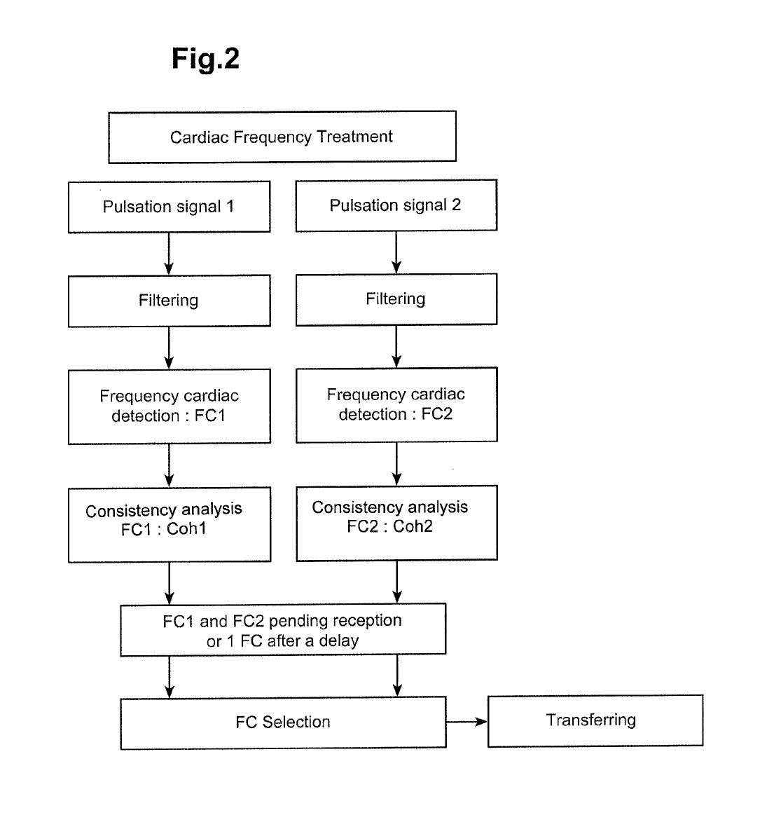

FIG. 2 shows a diagram representing different steps of the method for measuring heart rate,

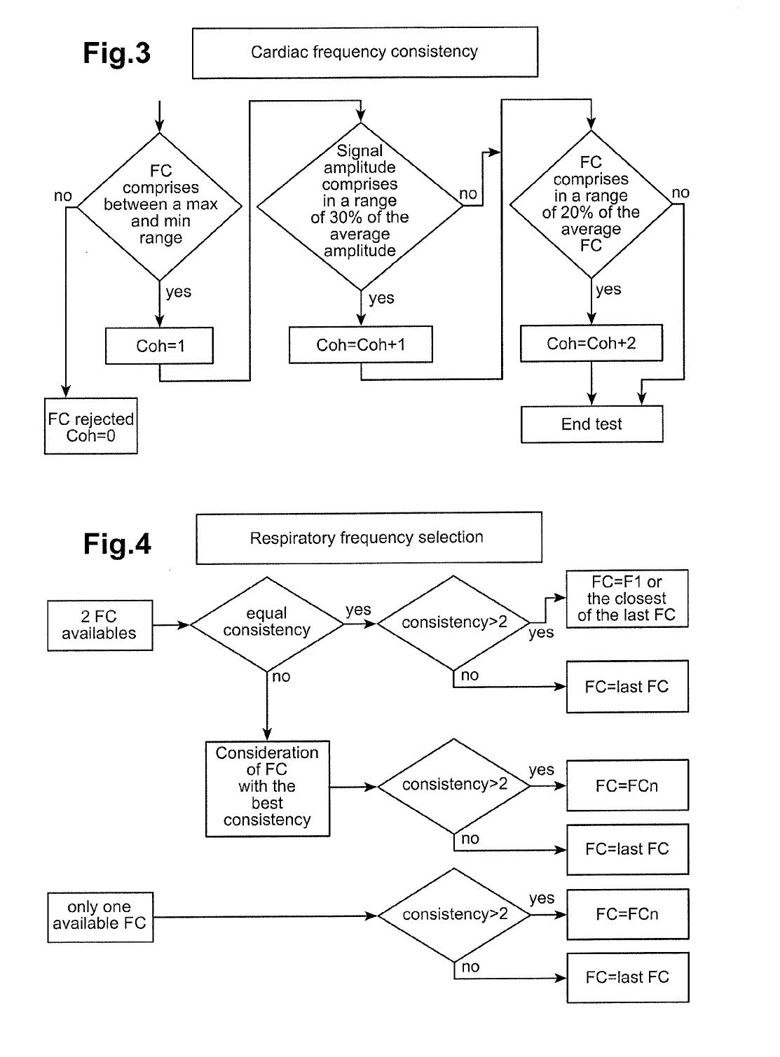

FIG. 3 shows a diagram of the different steps of evaluating the consistency of a heartbeat signal,

FIG. 4 shows a diagram of the different steps of selecting the new reference heartbeat,

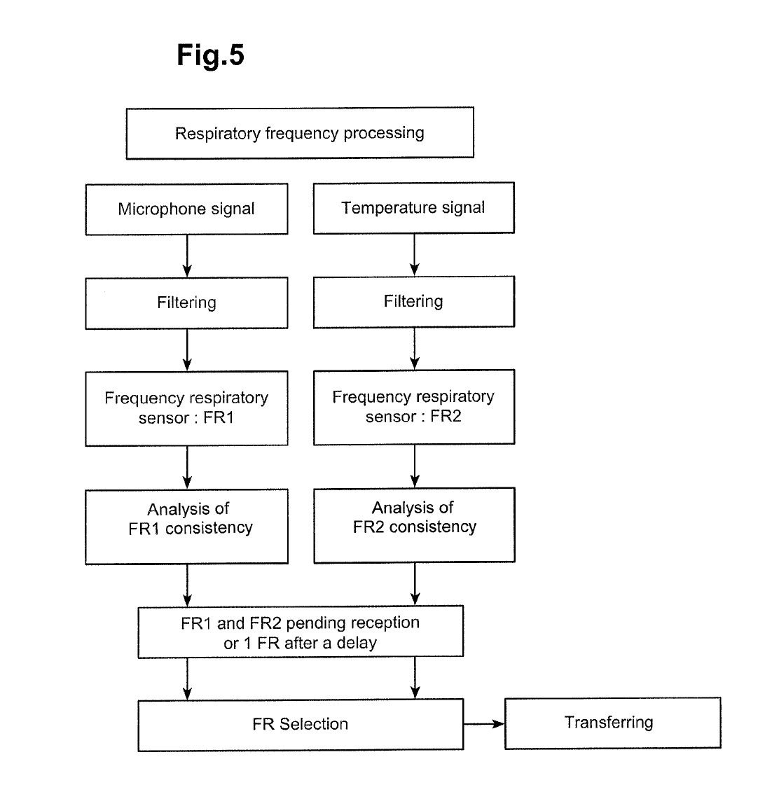

FIG. 5 shows a diagram representing different steps of the method for measuring respiratory rate,

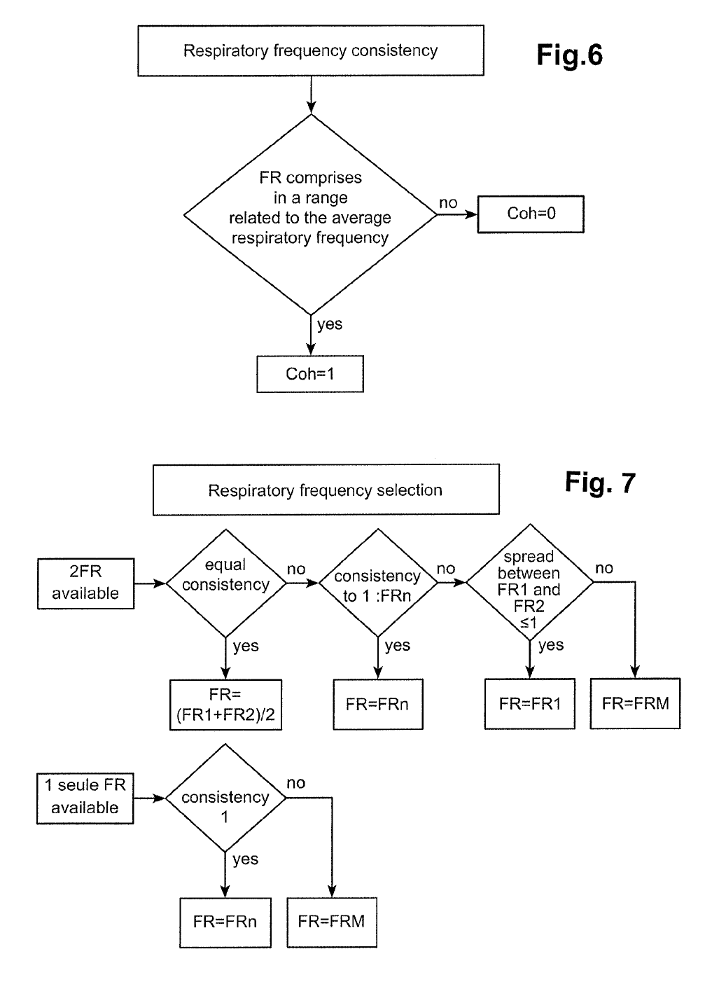

FIG. 6 shows a diagram of the different steps of evaluating the consistency of a respiratory rate signal,

FIG. 7 shows a diagram of the different steps of selecting the new reference respiratory rate.

By referring mainly to FIG. 1, one can see, represented diagrammatically, an example of embodiment of a measurement device 1 for measuring a biological rhythm. This example of embodiment allows a clear understanding of the invention; it is however important to note that both the device and the method are not restricted to measuring a biological rhythm, but also extend to measuring any physiological parameter and for example allow measurement of blood oxygen saturation or moreover blood pressure.

In the specific example described below, since a rate to be measured is involved, the measurement performed is that of a frequency; quite obviously, when other physiological parameters to be measured are involved, one may speak, depending on the case, of a level or furthermore of a percentage or moreover more generally of a value, whereby the principle of the measurement remains however completely identical.

This measurement device 1 allows measurement of two biological rhythms, i.e. heart rate and respiratory rate. In other conceivable embodiments, the device 1 will only allow measurement of a single biological rhythm. The measurement device 1 comprises two sensors for measuring a biological rhythm and consequently two sensors Cc1 and Cc2 for measuring heart rate and another two Cr1 and Cr2 for measuring respiratory rate.

In the example of embodiment in the appended figures, the two sensors Cc1 and Cc2 are identical; pulse sensors or oximeters are involved. The other two sensors Cr1 and Cr2 are on the other hand different; i.e. a sensor Cr1 consisting of a microphone and a sensor Cr2 consisting of a temperature detector.

Use of two different sensors for measuring a biological rhythm offers the advantage of restricting the probability that the two sensors will not function simultaneously, all the more so in that both sensors are not sensitive to the same types of interference such as vibrations, impacts or furthermore temperature and relative humidity.

The measuring method associated with the device 1 may however process at the same time different types of, or identical sensors in order to obtain reliable measurements. In the case of identical sensors, provision may advantageously be made for different placing or positioning of the sensors such that although identical, interference with one of the sensors will not mean interference with the others of the same type.

The measurement device 1 furthermore comprises filtering means 2 of the signals emitted by sensors Cc1, Cc2, Cc1 and Cc2. These filtering means 2 allow elimination of the parasite peaks related to the signals transmitted by the sensors and transformation of these signals in order to be able to process the latter. The measurement device 1 also comprises the processing means 3 of the measured data. Storage means 4 of the data and display means 5 for the frequency selected are associated with the processing means 3. Advantageously, the storage means 3 comprise a flash-type memory. The display means 5 advantageously consist of an LCD-type screen.

It is also important to note that in the example of embodiment, the measurement device 1 comprises data sending means 6 allowing remote location of the display means 5 or furthermore display the data directly or additionally on a central unit, not illustrated in the appended figures.

Furthermore, the measurement device 1 comprises in an advantageous variant alarm means allowing a warning when the reference frequency is outside a value range.

A first example of functioning of the measuring method for measuring heart rate will be described, referring in this instance to FIGS. 2 to 4.

As illustrated in the diagram in FIG. 2 corresponding to the steps of the process, the measurement is performed based on the two sensors Cc1 and Cc2. It is important to note however that the sensors may be greater in number and particularly under circumstances in which there is a high risk of losing the sensors or when reliability of the latter is limited.

The method involves performing a step of measuring the biological rhythm for each sensor Cc1, Cc2 allowing generation of a series of measurements of at least two frequencies Fc1 and Fc2. The method subsequently involves performing a step of evaluating the level of consistency of each frequency Fc1, Fc2 of the measurement series, This step is particularly important as it will allow elimination of the erroneous or suspect measurements and retention of the more consistent measurements.

FIG. 3 depicts a diagram illustrating an example of embodiment of the evaluation step. The evaluation step allows assignment of a level of consistency to each measurement of the series. To this end, the evaluation step comprises at least two eliminatory analyses in series in which it is verified whether the value of each frequency belongs to a value range. More specifically in this example, the evaluation step comprises three eliminatory analyses in series, wherein a successful analysis provides both a point of consistency at the frequency measured and continuation to the following analysis.

In these analyses, it is verified whether the frequency measured belongs to a value range. Hence, in the first analysis, it is studied whether the frequency Fc1 measured belongs to a value range included between a minimum and a maximum value; one continues to the second analysis if appropriate and a first point of consistency is assigned to the frequency Fc1.

The second analysis involves checking whether the signal has an amplitude within a range corresponding to a percentage of the mean signal amplitudes selected, known as MSA. The third analysis involves checking whether the frequency Fc1 has a value within a range corresponding to a percentage of the mean frequencies selected, known as MFS. The percentage applied is advantageously 30% for the second analysis and 20% for the third analysis. A successful second and third analysis allows allocation of a second point of consistency followed by a third if appropriate.

This evaluation step is performed for all the other frequencies of the series, i.e. the frequencies measured by the other cardiac sensors in this case Fc2.

When one of the frequencies Fc1 or Fc2 does not receive any consistency point, it will be eliminated during the subsequent selection step.

This selection step allows choice of a frequency among all the frequencies of the series as a function of their respective level of consistency on the one hand and a so-called reference frequency RF on the other hand, in order to determine a new reference frequency RF.

FIG. 4 depicts a diagram illustrating an example of embodiment of the step of selecting between the two frequencies Fc1 and Fc2 of a measurement series of both heart rate sensors Cc1 and Cc2.

This step comprises a comparison between the level of consistency of each frequency in the measurement series in order to retain only the frequencies displaying the highest level of consistency of the series. In the event that a frequency is unavailable, its level of consistency is considered to be zero.

The means of processing 3 subsequently select the closest frequency, known as CF, to the reference frequency, known as RF, among the frequencies displaying the highest level of consistency.

Once the frequency CF has been selected, the reference frequency RF is replaced by this frequency CF, or the reference frequency RF is maintained if the level of consistency of the closest frequency CF is below a threshold value.

Advantageously, the frequency CF will become the new reference frequency RF when the level of consistency achieved is greater than or equal to 2. This value may of course be modified, particularly as a function of the consistency points attributed following each analysis or moreover as a function of the number of analyses performed during the evaluation step.

The method also includes a step of storing the new reference frequency RF in the storage means 4.

A first example of functioning of the measuring method for measuring respiratory rate will be described, referring in this instance to FIGS. 5 to 7. As illustrated in the diagram in FIG. 5 corresponding to the steps of the method, the measurement is performed based on the two sensors Cr1 and Cr2. In this case also, the sensors may be greater in number and particularly under circumstances in which there is a high risk of loss or destruction of the sensors.

In the case of measurement of respiratory rate, the same steps are followed as for measurement of heart rate with however variants in the evaluation and selection step.

It is important however to remember that this example is non-restrictive and that variants of embodiment of both these evaluation and selection steps are conceivable.

Consequently, this measurement of respiratory rate includes a step of measuring the biological rhythm for each sensor Cr1, Cr2 allowing generation of a series of measurements of at least two frequencies Fr1 and Fr2. A step of evaluating the level of consistency of each frequency Fr1, Fr2 of the measurement series is subsequently performed.

The selection step among all the frequencies of the series as a function of their respective level of consistency on the one hand and a so-called reference frequency RF on the other hand is likewise performed.

FIG. 6 depicts a diagram illustrating an example of embodiment of the evaluation step. In the example, this step is considerably simplified in comparison to the step scheduled for measurement of heart rate. The purpose of this step is nevertheless identical, i.e. to allow allocation of a level of consistency to each measurement of the series.

To this end, the evaluation step comprises an eliminatory analysis in which it is verified whether the value of each frequency belongs to a value range. More specifically, it is checked whether the frequency Fr1 is included within a range corresponding to a percentage of the mean amplitude of the respiratory rates. This evaluation step is performed for all the other frequencies of the series, i.e. for the frequencies measured by the other respiratory sensors, i.e. in the example Fr2.

When one or both frequencies Fr1 or Fr2 does not receive any consistency point, it will be eliminated during the subsequent selection step.

Like the selection step for measuring a heart rate, the selection step allows choice of a frequency among all the frequencies of the series as a function of their respective level of consistency on the one hand and a so-called reference frequency RF on the other hand, in order to determine a new reference frequency RF.

FIG. 7 depicts a diagram illustrating an example of embodiment of the step of selecting between the two frequencies Fr1 and Fr2 of a measurement series of both sensors Cr1 and Cr2. This selection step comprises a comparison between the level of consistency of each frequency in the measurement series in order to retain only the frequencies displaying the highest level of consistency of the series.

The selection step subsequently comprises a calculation of the mean frequencies among the frequencies previously selected. The method subsequently consists of replacing the reference frequency (RF) with said new frequency, or of retaining the reference frequency (RF) if the level of consistency of the frequencies included in calculation of the mean frequency is below a threshold value.

In the event that a frequency Fr1 or Fr2 is unavailable, its level of consistency is considered to be zero.

The method also includes a step of storing the new reference frequency RF in the storage means 4, wherein the latter store both the reference frequency RF for heart rate and for respiratory rate.

The multi-sensor measurement device and the method according to the invention therefore make it possible to obtain, by associating a level of measurement consistency with a measurement of a sensor, following the frequency selection step, a reliable result for measurement of the biological rhythm observed.

Consequently, this measurement device is particularly suitable for measuring biological rhythms under difficult conditions and more generally, any physiological parameter requiring precise measurement, regardless of the circumstances of performing the measurements. Advantageously, this measurement device is to be incorporated in a helmet so as to be easily positioned and held on the individual.

Naturally, other embodiments and variants within the scope of the person skilled in the art could also have been envisaged, without forasmuch departing from the framework of the invention defined by the following claims.

1. Method for measuring a physiological parameter such as a biological rhythm, on the basis of at least two sensors, wherein the following are performed:
a step of measuring the physiological parameter for each sensor, allowing the generation of a series of measurements of at least two values,
a step of evaluating the level of consistency of each value of the measurement series,
a step of selecting a value from all the values of the series as a function of their respective level of consistency on the one hand and a so-called reference value on the other hand in order to determine a new reference value,
a step of storing the new reference value.

2. Method for measuring a physiological parameter according to claim 1, on the basis of at least two sensors, wherein measurement of a biological rhythm is performed with:
a step of measuring the biological rhythm for each sensor allowing generation of a series of measurements of at least two frequencies,
a step of evaluating the level of consistency of each frequency of the measurement series,
a step of selecting a frequency among all the frequencies of the series as a function of their respective level of consistency on the one hand and a so-called reference frequency on the other hand, in order to determine a new reference frequency,
a step of storing the new reference frequency.

3. Method of measuring a physiological parameter according to claim 2 wherein the evaluation step comprises at least two eliminatory analyses in series in which it is verified whether the value of each frequency belongs to a value range, wherein belonging to the range increases the level of consistency of the frequency.

4. Method of measuring a physiological parameter according to claim 3 wherein the value range comprises a low value and a high value corresponding to a percentage of the mean amplitude of the frequencies selected (MFS).

5. Method of measuring a physiological parameter according to claim 2, wherein the following are performed during the selection step:
comparison between the level of consistency of each frequency in the measurement series in order to retain only the frequencies displaying the highest level of consistency of the series,
selection of the closest frequency to the reference frequency among the frequencies displaying the highest level of consistency,
replacement of the reference frequency by the frequency with the closest value, or maintenance of the reference frequency if the level of consistency of the closest frequency is below a threshold value.

6. Method of measuring a physiological parameter according to claim 2, wherein the following are performed during the selection step:
comparison between the level of consistency of each frequency in the measurement series in order to retain only the frequencies displaying the highest level of consistency of the series,
the mean frequency among the frequencies displaying the highest level of consistency,
replacement of the reference frequency (RF) with said mean frequency, or maintenance of the reference frequency (RF) if the level of consistency of the frequencies included in calculation of the mean frequency is below a threshold value.

7. Device for measuring a physiological parameter such as a biological rhythm for implementing the method according to claim 1, comprising at least two sensors for measuring a physiological parameter, means of filtering the signals issued by the sensors, means of processing the measured data, means of storing the data and means of displaying the selected value.

8. Device for measuring a physiological parameter such as a biological rhythm according to claim 7 wherein at least two sensors of the measurement of a physiological parameter are of different technologies.

9. Device for measuring a physiological parameter such as a biological rhythm according to claim 7, comprising alarm means allowing a warning when the reference value is outside a value range.

10. Device for measuring a physiological parameter such as a biological rhythm according to claim 7, comprising at least four sensors and allowing measurement of the heart rate and respiratory rate of an individual.
